(19) United States
(12) Patent Application Publication
(10) Pub. No.: US 2004/0115808 A1
(43) Pub. Date: Jun. 17, 2004

(54) Enteric nervous system derived stem and progenitor cells and uses thereof

(76) Inventor: Vassilis Pachnis, London (GB)

Correspondence Address: GRAY CARY WARE FREDENRICH, 1625 MASSACHUSETTS AVENUE, NW, SUITE 300, WASHINGTON, DC, 20036-2247, US

(21) Appl. No.: 10/470,993

(22) PCT Filed: Feb. 2, 2002
(86) PCT No.: PCT/GB02/00440

(30) Foreign Application Priority Data
Feb. 2, 2001 (GB) 0102717.6

(51) Int. Cl. C12N 5/08

(52) U.S. Cl. 435/368

(57) ABSTRACT

Enteric nervous system derived multipotential progenitor cells (EPCs) (stem cells) can be isolated from postnatal mammalian gut tissue and maintained in culture in vitro. An in vitro cellular composition comprising enteric nervous system derived multipotential progenitor cells (EPCs), the multipotential progenitor cells being isolated from postnatal mammalian gut tissue, is described.

### TECHNICAL FIELD

The present invention relates generally to methods for isolating stem cells, particularly stem cells from the enteric nervous system, and their use in a human or other mammal.

### BACKGROUND TO THE INVENTION

For a very long time the prevailing dogma in neurobiology has been that the majority of cells of the nervous system are born during embryogenesis. However, a number of observations and work from several laboratories over the years has challenged that dogma. It is now known that, despite the fact that the majority of the cells of the nervous system are indeed born during embryogenesis and the early postnatal period, new neurons are continuously added in certain regions of the adult mammalian brain. This realisation has led to the development of protocols that allow the establishment of stem cell cultures from various regions of the adult brain. Such stem cells have the ability to self renew and are capable of generating neuronal and glial cells both in vitro and upon transplantation into the brain. Furthermore, these experiments raise the possibility that stem cells grown in culture can be used as a cell replacement therapy in various neurodegenerative diseases such as Parkinson's and Alzheimer's disease or congenital neural deficiencies (Momma et al., 2000; Temple and Alvarez-Buylla, 1999).

In addition to the central nervous system (CNS), self-renewing and multipotential stem cells have been isolated from the progenitors of the peripheral nervous system (PNS) in embryos. Thus, Stemple and Anderson showed several years ago that neural crest stem cells (NCSCs) can be established from explants of neural tube of E10.5 rat embryos. NCSCs can be maintained as multipotential cells for extended periods and depending on the culture conditions they can differentiate into neuronal and glial cells (Stemple and Anderson, 1992). Interestingly, similar NCSC cultures have also been established from postmigratory neural crest derivatives that have colonised the sciatic nerve of E15 rat embryos (Morrison et al., 1999). These findings suggest that multipotential progenitors of the PNS are present in mammalian embryos at relatively late stages of PNS histogenesis.

The enteric nervous system (ENS) of vertebrates is the most complex part of the PNS. It is composed of a large number of diverse types of neurons and glial cells organised into plexi of interconnected ganglia arranged as two concentric rings around the radial axis of the gut wall, the outer myenteric and the inner submucosa (Gershon et al., 1994). As is the case for most cells of the PNS, the ENS is derived entirely from neural crest (NC) cells (Le Douarin and Teillet, 1973). The majority of the progenitors of the ENS are generated at the vagal NC of the postotic hindbrain at the level of somites 1-7. Shortly after delamination from the neural tube (E8.5-9.0), a subpopulation of vagal NC cells migrates ventrolaterally and accumulates in the immediate postbranchial region, ventrally to the cervical branches of the dorsal aorta, where, under the influence of local signals, the vagal NC cells induce expression of the RET tyrosine kinase receptor (RT.K) (Durbec et al., 1996). The RET+ vagal NC cells invade the foregut mesenchyme (enteric neural crest (ENC) cells) and, migrating in a rostrocaudal direction, colonise over a period of 3-4 days (E9.5-13.5) the entire length of the gut and generate the majority of neurons and glia of the ENS (Durbec et al., 1996; Kapur et al., 1992).

Over the last several years, a number of molecules have been identified that play an important role in the development of the ENS in mammals. Among them are the neurotrophic factor Glial Cell Line-derived Neurotrophic Factor (GDNF) and its receptors, GFRα-1 (co-receptor) and Ret (signalling receptor). Other molecules important for ENS development are the Endothelin-3 (ET-3) and its receptor EDNRB and the transcription factors Mash-1, Phox2B and Sox10 (Taraviras and Pachnis, 1999). Work in the inventors laboratory has focused on understanding the role of the GDNF-RET signal transduction pathway in the development of the mammalian ENS. Several years ago the present inventors demonstrated that mice carrying a null mutation of c-Ret lack all enteric ganglia posterior to the oesophagus (intestinal aganglionosis)(Durbec et al., 1996; Schuchardt et al., 1994). These findings are consistent with genetic analysis in humans which has shown that individuals heterozygous for loss-of-function mutations in c-RET often develop congenital megacolon (Hirschsprung's disease), a condition characterised by absence of enteric ganglia from parts of the colon (Parisi and Kapur, 2000).

To further understand the development of the mammalian ENS in general and the mechanism of action of the RET RTK in particular, the present inventors have also developed and used an organotypic culture system of mouse fetal gut (Natarajan et al., 1999). At the stage of culture initiation, the gut is partially populated by undifferentiated ENS progenitors, but culturing for several days results in extensive neuronal and glial cell differentiation. Using this culture system, the development of the ENS in wild-type and RET-deficient gut have been compared, showing that the aganglionic phenotype observed in vivo is consistently reproduced under the in vitro culture conditions. Furthermore, microinjection of ENC cells isolated from the bowel of E11.5 mouse embryos into wild-type or RET-deficient aganglionic gut in organ culture, results in extensive re-population of the gut wall. Finally, using a similar approach, it has been demonstrated that single ENC cells introduced into the wall of wild-type gut generate both cell lineages of the ENS, i.e. neurons and glia. These data show that the ENC cells of E11.5 mouse embryos constitute a multipotential cell population that is capable of colonising efficiently both wild-type and aganglionic bowel in organ culture (Natarajan et al., 1999).

To date, multipotent stem cells have therefore been available from embryonic PNS and adult and embryonic CNS tissues. Although such cells can be used in cell replacement therapies, their use is severely limited by a number of factors. Due to the difficulty of accessing CNS tissue, isolation of such stem cells from the CNS can only be performed using embryonic tissue or adult tissue isolated at post-mortem. Such tissue is therefore difficult to isolate and, moreover, cell replacement therapies using such tissue may be associated with problems relating to immune rejection. Similar problems are associated with embryonically derived PNS stem cells.

### DISCLOSURE OF THE INVENTION

The present inventors have surprisingly discovered that, enteric nervous system derived multipotential progenitor cells (EPCs)(stem cells) can be isolated from postnatal mammalian gut tissue and maintained in culture in vitro. The mammalian gut can therefore provide a novel and easily accessible source of multipotential progenitor cells of the ENS, which overcome some of the problems associated with the prior art.

Therefore according to a first aspect of the present invention there is provided an in vitro cellular composition comprising enteric nervous system derived multipotential progenitor cells (EPCs) wherein said multipotential progenitor cells are isolated from postnatal mammalian gut tissue.

In a second aspect of the invention, there is provided a method of obtaining a source of enteric nervous system derived multipotential progenitor cells comprising:

(a) obtaining a sample of gut muscle from a postnatal mammal,

(b) disrupting said sample to obtain isolated cells from the sample,

(c) culturing the cells obtained from said disruption in culture medium, and

(d) maintaining said culture or disrupting said cell layers and culturing in fresh culture medium until a substantially pure sample of enteric nervous system derived multipotential progenitor cells is obtained.

By substantially pure it is meant that a majority of the cells, preferably greater than 60%, 70%, 80%, 90%, 95%, 98% or 99% of cells are ENS multipotent progenitor cells.

This method can be used to obtain cells from an animal for transplant into humans or may be practiced on the human body in order to obtain cells from the patient in need of treatment for autologous transplant.

In a further aspect of the invention, there is provided a method of obtaining a source of enteric nervous system derived multipotential progenitor cells comprising:

(a) providing a sample of gut muscle from a postnatal mammal,

(b) disrupting said sample to obtain isolated cells from the sample,

(c) culturing the cells obtained from said disruption in culture medium, and

(d) maintaining said culture or disrupting said cell layers and culturing in fresh culture medium until a substantially pure sample of enteric nervous system derived multipotential progenitor cells is obtained.

This is the first demonstration that such cells exist postnatally in the gut. Such cells may therefore provide an easily accessible source of multipotential progenitor cells of the ENS, which after culturing, may be used in the treatment of a variety of diseases.

Therefore, in a further aspect of the invention, there is provided a composition comprising enteric nervous system derived multipotential progenitor cells (EPCs) derived from postnatal mammalian gut tissue for use in a method of treatment of the human or animal body.

Also provided is the use of enteric nervous system derived multipotential progenitor cells (EPCs) derived from postnatal mammalian gut tissue for the manufacture of a medicament for use in a method of treatment, including the treatments as described herein.

The invention also provides a method of treatment of the human or animal body as described herein, said method including delivering to the subject in need of treatment an effective amount of cells to bring about amelioration of disease. By amelioration is meant curing of disease or relief (wholly or partially, permanently or temporarily) from one or more symptoms of disease.

Accordingly, the present invention provides compositions comprising the above cellular compositions or cells in admixture with a suitable carrier. In this aspect, the present invention provides pharmaceutical compositions suitable for delivering said cells for subsequent implant into a patient. In a further aspect, the present invention provides pharmaceutical compositions comprising the cells as obtainable using the above methods.

In a further aspect, the present invention provides the above pharmaceutical compositions for use in methods of medical treatment.

### DETAILED DESCRIPTION

EPC cells may be identified and discriminated from other gut cells both morphologically and by the presence of immunological markers expressed in EPC cells. For example, during embryogenesis, the multipotential progenitors of the ENS express Ret (Pachnis et al, 1993, Durbec et al., 1996; Tsuzuki et al., 1995) and the inventors have found that this receptor is also expressed in the ENS cells postnatally. Other markers which may be used to identify the ENS derived cells include PGP9.5 (Schofield et al, 1995) and Tuj1 (Ferreira and Caceres, 1992)(which are expressed in postmitotic neurons) and GFAP (Jessen and Mirsky, 1980)(which identify mature glial cells). As described below, the inventors have found that the majority of the ENS cells express both GFAP and Tuj1 with the remaining cells expressing either GFAP or Tuj1. Morphologically, the cultured multipotential progenitors of the ENS are flat triangular cells with short processes, which may extend up to 2-3 cell diameters in length.

The gut tissue to be used in the present invention may be obtained from any mammal, which may be human or non-human, such as rabbit, guinea pig, rat, mouse or other rodent, cat, dog, pig, sheep, goat, cattle or horse. Preferred animals-are rodent, such as mouse or rat, preferably mouse.

The tissue may be isolated at any time after birth. In a preferred embodiment, the cells are isolated from mammals up to 15 days old. However, the cells may be isolated from older mammals, for example 30, 50, 100 days old, including adult mammals.

To obtain cells according to the invention a section of gut can be obtained from a subject animal or a human patient undergoing surgery. The tissue sample may then be dissected to isolate the ganglia of the myenteric plexus and digested with enzymes such as trypsin and collagenase, e.g. collagenase at 0.5 mg/ml, to dissociate the cells of the section. The choice and concentration of enzymes for isolation of the cells will depend on the type, size and age of tissue used and will be within the knowledge of the skilled person. The dissociated tissue is then plated onto tissue culture plates under appropriate conditions. In a preferred embodiment, the tissue is plated onto fibronectin coated tissue culture plates in medium containing 15-20% chicken embryo extract (Morrison et al 1999). These culture conditions selectively promote the proliferation of multipotential enteric nervous system progenitors which, in approximately 7 days, are the major cell type present in the culture plate.

The cells resulting from the proliferation of multipotential enteric nervous system progenitors can be used to produce clonal cultures which clearly show that single cells derived from postnatal mammalian ENS can generate, in vitro, both neurons and glial cells. Cultures of multipotential progenitor cells, such as those described above, contain neurons expressing specific neuropeptides [such as Neuropeptide Y (NPY), Calcitonin Gene Related Peptide (CGRP), Somatostatin (SOM), Vasoactive Intestinal Peptide (VIP)] or enzymes important for the biosynthesis of specific neurotransmitters [Nitric Oxide Synthase (NOS)]. Using any one or all of NPY, CGRP, SOM, VIP or NOS as a series of molecular markers the inventors have shown that the culture conditions described above are capable of promoting the generation of neuronal subtypes that are normally present in the mature mammalian ENS.

The present invention therefore also envisages the direction of multipotential progenitor cells towards specific neuronal phenotypes by modification of the in vitro culture conditions. This aspect of the invention finds particular use in the generation of specific neurons suitable for transplantation into additional sites within the mammalian nervous system.

The cells of the invention may be maintained as a population without undergoing senescence for preferably at least 7, more preferably at least 14; yet more preferably at least 28, even more preferably at least 56 or most preferably at least 90 days. The chick embryo extract prevents differentiation of these cells.

A composition of the invention will preferably comprise at least 103 cells, more preferably at least 104 cells, even more preferably at least 105 cells; most preferably at least 106 cells; at least 90% of which are EPCs.

Cells and cell compositions of the invention may be used in autologous transplant i.e. to the individual from which the EPC cells were derived or for heterologous transplant i.e. to another individual. Autologous transplant is preferred in order to avoid problems with immune rejection of the cells. The cells and compositions of the present invention are particularly suitable for autologous transplant due to the large area of the gut, in particular the small intestine, allowing surgical removal of a small portion of tissue for isolation of the cells without any deleterious effect on the function of the organ. However, where the subject to be treated cannot be used as the source of EPC cells, heterologous transplant of cells may be used.

Treatment may be directed to cell replacement in parts of the enteric nervous system. For example, such cells and compositions of the invention may be used in cell replacement therapy to treat disorders in which particular cell types are missing from one part of the gut but present in other parts. For example, congenital megacolon (Hirschsprung's disease) is characterised by a lack of enteric ganglia from parts of the colon.

Recently, the present inventors have developed a mouse strain that expresses only one of the two RET isoforms which mimics the human condition in that enteric ganglia are absent only from a localised region of the large intestine (E. deGraaff et al., manuscript in preparation). Experimental analysis of this mouse model will provide a better understanding of the pathogenetic mechanisms of Hirschsprung's disease and will allow testing of therapeutic approaches for this neural deficiency that are based on cell replacement using the cells and compositions of the invention.

Other diseases and disorders of the gastrointestinal tract characterised by loss of neuronal cells or presence of defective neuronal cells at sites in the intestinal tract and in which the present invention may find use include, but are not limited to, intestinal pseudo-obstruction, achalasia, congenital defects, constipation, prematurity and conditions secondary to viral infection.

However, the compositions and cells of the invention may be further used in the treatment of diseases and disorders of the central nervous system or peripheral nervous system. For such treatment, the cellular composition comprising the progenitor cells is preferably cultured with growth factors required for induction of differentiation to the desired cell type. The invention may therefore be used in treatment of neurodegenerative diseases and neuronal disorders of the CNS. Such diseases and disorders include, but are not limited to, Parkinson's disease, Alzheimer's disease, congenital neural deficiencies, Huntingdon's chorea, and neuronal cell loss due to stroke or injury.

Where the cells and compositions of the invention are being used in methods of treatment, it is preferred that the cells and compositions are delivered to the appropriate tissue in the body. For example, where treatment is of a disease or condition of the colon e.g. Hirschsprung's disease, the cells or compositions should be directed to the colon. Where treatment is of a disease or condition of the brain e.g. Alzheimer's disease, the cells or compositions should be delivered to the brain.

In accordance with the present invention, compositions provided may be administered to individuals. Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Compositions of the present invention may be administered to a patient in need of treatment via any suitable route, for example by injection into the site to be treated. The precise dose will depend upon a number of factors, including the size and location of the area to be treated and the precise nature of the composition. The dose for a single treatment of an adult patient may be proportionally adjusted for children and infants. Treatments may be repeated at daily, twice-weekly, weekly or monthly intervals, at the discretion of the physician.

Cells of the present invention will usually be administered in the form of a pharmaceutical composition, which may comprise at least one component in addition to the multipotent progenitor cells.

Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration.

For injection at the location of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition may be administered alone or in combination with other treatments, either simultaneously or sequentially dependent upon the condition to be treated.

The invention will now be further described with reference to the following non-limiting FIGURE and Examples. Other embodiments of the invention will occur to those skilled in the art in the light of these.

### FIGURE

FIG. 1: shows enteric nervous system derived multipotential progenitor cells (EPCs) cultured from eight day old mice.

### EXAMPLES

### Example 1

Isolation of Enteric Nervous System Derived Multipotential Progenitor Cells (EPCS) from Postnatal Gut.

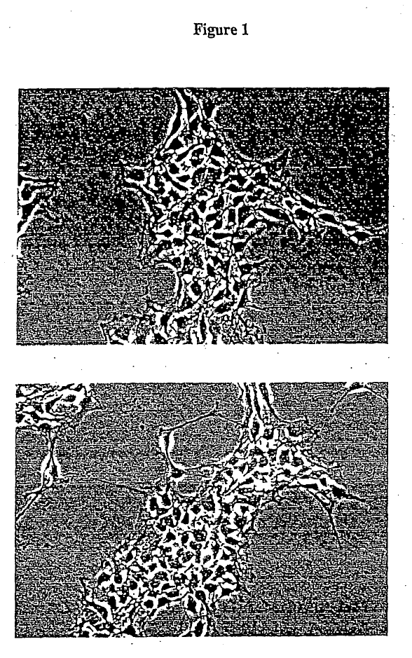

The intestine was removed from a postnatal mouse (P 2-15). The organ was removed and washed extensively with 1×PBS containing antibiotics (Penicillin/Streptomycin). Using forceps the external muscle layers of the organ which contain the enteric ganglia were then peeled-off. The tissue was subsequently incubated in collagenase (in 1×PBS) at 37° C. for approximately 30-45 minutes depending on the age of the animal. The dissociated tissue was then plated onto fibronectin coated tissue culture plates in medium containing 15-20% chicken embryo extract (Morrison et al 1999). The culture medium has the following composition (DMEM 85%, Chicken embryo extract (15%) (Gibco, UK), bovine fibroblast growth factor (20 ng/ml/hr) (Sigma, UK), N2 supplement (1%) (Life Technologies Ltd, UK), B27 Supplement (2%)(Life Technologies Ltd, UK), penicillin/streptomycin (1%), mercaptoethanol (50 μM), retinoic acid (35 ng/ml) (Sigma, UK). The presence of chick embryo extract at this concentration in the culture medium promote selectively the proliferation of multipotential enteric nervous system progenitors which, in approximately 7 days, are the major cell type present in the culture plate. After several days and passages, the cultures consisted of a homogeneous population of flat and short processes-bearing cells (FIG. 1) that were reminiscent of primary neural crest stem cells (NCSCs) described by Stemple and Anderson, 1992). We were able to expand and propagate the NCSC-like cultures considerably: starting from small segments of the small intestine derived from 2-3 animals we generated several millions of cells which could be maintained as a highly proliferating cell population without undergoing senescence for at least 90 days. Therefore, these findings suggest that our culture conditions support the proliferation and expansion of a cell population that is derived from enteric ganglia and can generate mature enteric neurons and glial cells.

### Example 2

Identification of EPC Cells

To confirm that these cells were derived from the enteric ganglia and thus were of neural crest origin, similar cultures were established from the gut of Ret-deficient (Ret.k-/Ret.k-) newborn (P1) animals, which lack enteric ganglia from the entire small and large intestine. No NCSC-like cells appeared in cultures established from Ret.k-homozygous animals, strongly suggesting that the characteristic cell population is derived from enteric ganglia.

To further characterise this ENS-derived cell population, the NCSC-like cell population were stained with a series of molecular markers that are expressed in mature neurons and glial cells of the ENS. Among the molecular markers used were PGP9.5 and Tuj1 (expressed in postmitotic neurons) and GFAP (which identifies mature glial cells). The majority of cells (60%) expressed bbth GFAP and Tuj1 suggesting that these double positive cells represent uncommitted multipotential progenitors of the mature neurons and glia of the mammalian ENS. In addition to these double positive cells, smaller fractions of GFAP+-only (25%) and Tujl+-only (15%) cells were also present in these cultures.

Since during embryogenesis the multipotential progenitors of the ENS express Ret, the putative EPC cultures were examined for expression of this tyrosine kinase receptor using immunostaining and FACS analysis using a FACStar (Becton-Dickinson) Fluorescence Activated Cell Sorter with an Argon ion laser at 488 nm using the methods described in Natarajan et al, 1999 and Lo and Anderson, 1995. Anti-Ret antibodies (Young et al, 1999) were obtained from ABL, Japan.

The experiments show that the vast majority of the cells established from postnatal gut express the Ret RTK, a characteristic marker of enteric nervous system derived multipotential progenitor cells. The morphology and marker analysis strongly suggest that the EPC cells represent multipotential progenitors of the mammalian ENS.

### Example 3

Differentiation of EPC Cells in the Presence of Neurotrophic Factors

In order to further test that the cultured cells are Ret+ cells, the cells were cultured with the Ret ligands GDNF and nurturing (Ballot et al, 2000) (After 4 days culturing in the presence of 10 ng/ml of GDNF, it was found that the majority of cells exhibited long axonal processes and expressed high levels of PGP9.5 and Tuj1. Similar results were obtained after culturing with 10 ng/ml of neurturin.

### Example 4

Migration and Differentiation of EPC Cells from Mouse Postnatal Gut Injected into E8.5 Embryos

For these experiments, postnatal EPCs are isolated using the method of Example 1 but from transgenic mice which express ubiquitously the bacterial lacZ gene, for example PTY mice (kindly provided by Dr. R. Beddington, NIMR). Alternative. strains of mice which express ubiquitously the bacterial lacZ gene and which may be used include the Rosa26 strain of mice.

E8.5 mouse embryos of a non-lacZ expressing strain are dissected from the uterus with the intact visceral yolk sac and cultured in vitro for 24 hours in DR50 medium (50% DMEM, 50% rat serum) in 5% O2, 5% CO2, 90% N2, atmosphere and for a further 48 hours in DR75 medium (25% DMEM, 75% rat serum) in 20% O2, 5% CO2, 75% N2, atmosphere according to the method of Sturm and Tam (1993). Postnatal EPCs of a lacZ expressing strain (as described above) are then transplanted in the pathway of migration of vagal neural crest cells i.e. lateral to the neural tube at the level of somites 1-5 using the method as described in Trainor and Krumlauf (2000). After 3 days culture, the migration and the differentiation of the cells in vivo can be examined by β-galactosidase histochemistry.

For X-gal staining, tissues are fixed in 1% formaldehyde, 0.1% glutaraldehyde, 1 mM MgCl2, 1 mM EGTA and 0.02% NP40 (in PBS) at the end of the culture period for 20 minutes at 40 C. □-galactosidase-expressing cells are visualised by incubating tissue samples at 370 C (O/N) in staining solution containing 5 mM potassium ferricyanide, 5 mM potassium ferrocyanide, 2 mM MgCl2, 0.01% deoxycholate and 0.02% NP40 and 1 mg/ml X-gal in PBS. The whole-mount preparations are photographed and then postfixed in 4% paraformaldehyde (in PBS) for 2 hours at room temperature, incubated in 30% sucrose (in PBS) O/N at 40 C, embedded in OCT compound and sectioned (20 □m) in a cryostat (Jung). The stained cells can then be identified both morphologically and using immunostaining to identify cell types e.g. PGP9.5 and/or Tuj1 to identify post-mitotic neurons and GFAP to identify mature glial cells.

The experiment is then repeated using a Ret− embryo in order to demonstrate that the postnatal EPCs may colonise Ret− deficient aganglionic gut wall.

### Example 5

Generation of Non-Enteric Neurons from EPC Cells

Lhx6 and Lhx7, a pair of closely related transcription factors of the LIM-homeodomain subclass, have recently been identified by the present inventors. These transcription factors are characteristically expressed in interneurons of the telencephalon (Grigoriou et al., 1998; Lavdas et al., 1999). More specifically, Lhx7 is expressed specifically in the cholinergic neurons of the basal ganglia, a population of cells that is affected in cases of Alzheimer's disease. Loss of function studies have further established that mice deficient in Lhx7 lack the majority of cholinergic neurons, suggesting that this transcription factor is necessary and sufficient for the differentiation of basal ganglia precursors to the cholinergic lineage (C. Hearn and V. Pachnis, unpublished observations). In addition, explant culture experiments have suggested that Lhx7 is induced in neuronal precursors by the growth factor FGF8 (Tucker et al., 1999).

Therefore, in order to test the extent to which EPCs can adopt the cholinergic properties of telencephalic neurons, EPCs are transferred to medium with low concentration of chicken embryo extract (1%) on plates coated with poly-D-lysine and laminin and are exposed in culture to the growth factor FGF8 at 10 ng/ml. After 4 days culturing in the presence of FGF8, the cultured cells are analysed by immunostaining for expression of Lhx7 (Grigoriou et al, 1998) using specific antibodies. The presence of Lhx7 indicates that EPCs can adopt the cholinergic properties of telencephalic neurons.

Alternatively, the experiment may be performed using EPCs derived from a mouse strain which expresses ubiquitously LacZ (encoding β-galactosidase), as described in Example 4, but under the control of Lhx7 regulatory sequences. This strain is derived by mutagenesis using targeted homologous recombination of the Lhx7 locus in mouse embryonic stem cells by standard protocols known to the skilled man. Cells which adopt cholinergic properties are identified by X-gal staining as described above.

### Example 6

Generation of Neurosphere-Like Bodies from EPCs.

EPCs were generated as described in Example 1. The formation of neurosphere-like bodies (NLBs) was promoted by the addition of epidermal growth factor in the EPC cultures 5 days after gut dissociation. The NLBs were shown to be composed of varying numbers of EPC cells which adhered to each other in spherical formations that detached from the tissue culture substrate and continued to grow as floating bodies these EPC-derived NLBs were similar in appearance to neurospheres formed upon culture of adult brain cells. Immunostaining of NLBs with generic neuronal and glial markers (TuJ1 and GFAP, respectively) revealed extensive differentiation of EPC cells towards the neuronal and glial cell lineages. Furthermore, immunostaining with neuronal subtype-specific markers, such as NPY, CGRP and VIP, indicated the presence of specific subtypes of neurons normally present in various parts of the peripheral and central nervous system. The ability to grow such cells in vitro could provide an efficient means of generating a diverse array of mature neuronal cell types.

References

Baloh, R. H., Enomoto,. H., Johnson E. M. Jr, Milbrandt J. (2000). The GDNF family ligands and receptors—implications for neural development. Curr. Opin. Neurobiol. 10(1), 103-110.

Durbec, P. L., Larsson-Blomberg, L. B., Schuchardt, A., Costantini, F., and Pachnis, V. (1996). Common origin and developmental dependence on c-ret of subsets of enteric and sympathetic neuroblasts. Development 122, 349-58.

Ferreira A. & Caceres (1992). Expression of the class III beta-tubulin isotype in developing neurons in culture. J. Neurosc. Res. 32(4), 516-29.

Gershon, M. D., Kirchgessner, A. L., and Wade, P. R. (1994). Functional anatomy of the enteric nervous system. In “Physiology of the Gastrointestinal Tract” (e. a. L. R. Johnson, Ed.), Vol. 1, pp. 381-422. Raven Press, New York.

Grigoriou, M., Tucker, A. S., Sharpe, P. T., and Pachnis, V. (1998). Expression and regulation of Lhx6 and Lhx7, a novel subfamily of LIM homeodomain encoding genes, suggests a role in mammalian head development. Development 125, 2063-74.

Jessen K. R. and Mirsky R. (1980). Glial cells in the enteric nervous system contain glial fibrillary acidic protein. Nature 286(5774), 736-7.

Kapur, R. P., Yost, C., and Palmiter, R. D. (1992). A transgenic model for studying development of the enteric nervous system in normal and aganglionic mice. Development 116, 167-75.

Lavdas, A. A., Grigoriou, M., Pachnis, V., and Parnavelas, J. G. (1999). The medial ganglionic eminence gives rise to a population of early neurons in the developing cerebral cortex [In Process Citation]. J Neurosci 19, 7881-8.

Le Douarin, N. M., and Teillet, M. A. (1973). The migration of neural crest cells to the wall of the digestive tract in avian embryo. J Embryol Exp Morphol 30, 31-48.

Lo L. & Anderson D. J. (1995). Postmigratory neural crest cells expressing c-Ret display restricted developmental and proliferative capacities Neuron 15(3), 527-39

Momma, S., Johansson, C. B., and Frisen, J. (2000). Get to know your stem cells. Curr Opin Neurobiol 10, 45-9.

Morrison, S. J., White, P. M., Zock, C., and Anderson, D. J. (1999). Prospective identification, isolation by flow cytometry, and in vivo self-renewal of multipotent mammalian neural crest stem cells [In Process Citation]. Cell 96, 737-49.

Natarajan, D., Grigoriou, M., Marcos-Gutierrez, C. V., Atkins, C., and Pachnis, V. (1999). Multipotential progenitors of the mammalian enteric nervous system capable of colonising aganglionic bowel in organ culture. Development 126, 157-68.

Pachnis V., Mankoo, S. B. and Costantini, F. (1993) Expression of the c-ret proto-oncogene during mouse embryogenesis. Development 119, 1005-1017

Parisi, M. A., and Kapur, R. P. (2000). Genetics of Hirschsprung disease [In Process Citation]. Curr Opin Pediatr 12, 610-7.

Pham, T. D., Gershon, M. D., and Rothman, T. P. (1991). Time of origin of neurons in the murine enteric nervous system: sequence in relation to phenotype. J Comp Neurol 314, 789-98.

Schofield J. N., Day I. N., Thompson R. J., Edwards Y. H. (1995). PGP9.5, a ubiquitin C-terminal hydrolase; pattern of mRNA and protein expression during neural development in the mouse. Brain Res Dev Brain Res. 85(2), 229-38

Schuchardt, A., V, D. A., Larsson-Blomberg, L., Costantini, F., and Pachnis, V. (1994). Defects in the kidney and enteric nervous system of mice lacking the tyrosine kinase receptor Ret [see comments]. Nature 367, 380-3.

Stemple, D. L., and Anderson, D. J. (1992). Isolation of a stem cell for neurons and glia from the mammalian neural crest. Cell 71, 973-85.

Taraviras, S., and Pachnis, V. (1999). Development of the mammalian enteric nervous system. Curr Opin Genet Dev 9, 321-327.

Temple, S., and Alvarez-Buylla, A. (1999). Stem cells in the adult mammalian central nervous system. Curr Opin Neurobiol 9, 135-41.

Tsuzuki, T., Takahashi, M., Asai, N., Iwashita, T., Matsuyama, M., and Asai, J. (1995). Spatial and temporal expression of the ret proto-oncogene product in embryonic, infant and adult rat tissues. Oncogene 10, 191-8.

Tucker, A. S., Yamada, G., Grigoriou, M., Pachnis, V., and Sharpe, P. T. (1999). Fgf-8 determines rostral-caudal polarity in the first branchial arch. Development 126, 51-61

Young H. M. Ciampoli D., Hsuan J., Canty A. J. (1999 Expression of Ret−, p75 (NTR)-, Phox2a, Phox2b-, and tyrosine hydroxylase-immunoreactivity by undifferentiated neural crest-derived cells and different classes of enteric neurons in the embryonic mouse gut. Dev. Dyn. 216(2), 137-52.

1. An in vitro cellular composition comprising enteric nervous system derived multipotential progenitor cells (EPCs). wherein said multipotential progenitor cells are isolated from postnatal mammalian gut tissue.

2. The in vitro cellular composition according to claim 1 wherein the enteric nervous system derived multipotential progenitor cells express the Ret tyrosine kinase receptor.

3. The in vitro cellular composition according to claim 1 or claim 2 wherein the enteric nervous system derived multipotential progenitor cells comprise at least one of PGP9.5, Tuj1 and GFAP molecular markers.

4. The in vitro cellular composition according to claim 3 wherein the enteric nervous system derived multipotential progenitor cells comprise Tuj1 and GFAP molecular markers.

5. A method of obtaining a source of enteric nervous system derived multipotential progenitor cells comprising:
(a) obtaining a sample of gut muscle from a postnatal mammal,
(b) disrupting said sample to obtain isolated cells from the sample,
(c) culturing the cells obtained from said disruption in culture,medium, and
(d) maintaining said culture or disrupting said cell layers and culturing in fresh culture medium until a substantially pure sample of enteric nervous system derived multipotential progenitor cells are obtained.

6. A method of obtaining a source of enteric nervous system derived multipotential progenitor cells comprising:
(a) providing a sample of gut muscle from a postnatal mammal,
(b) disrupting said sample to obtain isolated cells from the sample,
(c) culturing the cells obtained from said disruption in culture medium, and
(d) maintaining said culture or disrupting said cell layers and culturing in fresh culture medium until a substantially pure sample of enteric nervous system derived multipotential progenitor cells are obtained.

7. The method according to claim 5 or claim 6 further comprising:
(e) exposing the cultured cells to at least one growth factor specific for differentiation of CNS cells; and
(f) isolating differentiated cells.

8. The method according to claim 7 wherein the growth factor is FGF8 and the differentiated cells express Lhx7.

9. The in vitro cellular composition according to any one of claims 1 to 4 or the enteric nervous system derived multipotential progenitor cells obtained according to the method according to any one of claims 5 to 8 for use in a method of treatment of a human or animal body.

10. Use of the in vitro cellular composition according to any one of claims 1 to 4 or the enteric nervous system derived multipotential progenitor cells obtained according to the method of claim 5 or claim 6 in the preparation of a medicament for the treatment of Hirschsprung's disease.

11. Use of the in vitro cellular composition according to any one of claims 1 to 4 or the enteric nervous system derived multipotential progenitor cells obtained according to the method according to any one of claims 5 to 8 in the preparation of a medicament for the treatment of a neurodegenerative disease or a congenital neural disorder.

12. The use according to claim 11 wherein the disease or disorder is a disease or disorder of the central nervous system.
